(19) United States
(12) Patent Application Publication
(10) Pub. No.: US 2021/0282854 A1
(43) Pub. Date: Sep. 16, 2021

(54) A HAIR CUTTING DEVICE USING PULSED RADIATION

(71) Applicant: KONINKLIJKE PHILIPS N.V., EINDHOVEN (NL)

(72) Inventors: KIRAN KUMAR THUMMA, EINDHOVEN (NL); MARK THOMAS JOHNSON, ARENDONK (BE); RIEKO VERHAGEN, VUGHT (NL); BASTIAAN WILHELMUS MARIA MOESKOPS, UDEN (NL); MARIUS IOSIF BOAMFA, VELDHOVEN (NL)

(21) Appl. No.: 16/338,880

(22) PCT Filed: Oct. 9, 2017
(86) PCT No.: PCT/EP2017/075595

§ 371 Date: Apr. 2, 2019

(30) Foreign Application Priority Data
Oct. 18, 2016 (EP) 16194281.8

(51) Int. Cl. A61B 18/20

(52) U.S. Cl. CPC A61B 18/203; A61B 2018/00476

(57) ABSTRACT

There is provided a hair cutting device for cutting hair on a body of a subject. The hair cutting device comprises at least one light source for generating laser light at two or more specific wavelengths corresponding to wavelengths absorbed by one or more chromophores in hair; and a cutting element comprising an optical waveguide for receiving light from the at least one light source. The optical waveguide comprises a cutting face, the cutting face being arranged to contact hair as the hair cutting device is moved across the skin of the body of a subject. The cutting face is arranged essentially parallel to the long axis of the optical waveguide. The optical waveguide is arranged to allow the light generated by the at least one light source to couple into hair when hair is close to or in contact with the optical waveguide. The at least one light source is configured to generate laser light having a first wavelength and a series of pulses of laser light having a second wavelength. A method of operating a hair cutting device is also disclosed.

### FIELD OF THE INVENTION

The invention relates to a hair cutting device for cutting (e.g. shaving) hair on a body of a subject and, in particular, a hair cutting device which uses pulsed radiation. The invention also relates to a method of operating such a hair cutting device.

### BACKGROUND OF THE INVENTION

Shaving devices for cutting or shaving hair on a body of a subject typically make use of one or more blades that cut hairs as the device is moved across the skin of the subject. The blades can be static within the device, for example as in a wet razor, whereas in other types of devices, for example electric shavers, one or more blade elements can be actuated (e.g. rotated or oscillated) in order to produce a cutting action.

However, an alternative type of shaving device has been proposed in WO 2014/143670 that makes use of laser light. In particular, a laser light source is provided that is configured to generate laser light having a wavelength selected to target a predetermined chromophore to effectively cut a hair shaft. A fibre optic is located on a shaving portion of the device that is positioned to receive the laser light from the laser light source at a proximal end, conduct the laser light from the proximal end toward a distal end, and emit the light out of a cutting region of the fibre optic and toward hair when the cutting region is brought in contact with the hair.

### SUMMARY OF THE INVENTION

In order to cut or melt hair, sufficient optical energy needs to be delivered through a cutting element of the cutting device. However, in some scenarios, too much optical energy coupling out from the optical waveguide into hair may cause the temperature of the optical waveguide to increase to such a level that the optical waveguide may be damaged.

Thus, there exists a need for a hair cutting device in which the amount of high-powered, or potentially damaging light propagating through the optical waveguide is limited.

According to a first aspect, there is provided a hair cutting device for cutting hair on a body of a subject. The hair cutting device comprises at least one light source for generating light at two or more specific wavelengths corresponding to wavelengths absorbed by one or more chromophores in hair; and a cutting element comprising an optical waveguide for receiving light from the at least one light source. The optical waveguide comprises a cutting face, the cutting face being arranged to contact hair as the hair cutting device is moved across the skin of the body of a subject. The cutting face is arranged essentially parallel to the long axis of the optical waveguide. The optical waveguide is arranged to allow the light generated by the at least one light source to couple into hair when hair is close to or in contact with the optical waveguide. The at least one light source is configured to generate light having a first wavelength and a series of pulses of light having a second wavelength. A combination of light having one wavelength and pulsed light having a different wavelength provides advantages in terms of the longevity of the shaving device. Light at the two different wavelength may be effective at cutting different parts of a strand of hair, and the temperature increase of the optical waveguide caused melting hair may be low enough to avoid damage.

In some embodiments, the hair cutting device may comprise a first light guiding element configured to guide light from the at least one light source into a first end of the cutting element. The hair cutting device may further comprise a second light guiding element configured to guide light from the at least one light source into a second end of the cutting element. The first light guiding element may comprise a taper transition section in which a diameter of the light guiding element reduces from a first diameter to a second diameter. The second light guiding element may comprise a taper transition section in which a diameter of the light guiding element reduces from a third diameter to a fourth diameter. The third diameter may be the same as the first diameter. The fourth diameter may be the same as the second diameter. The light guiding element(s) may form a part of the optical waveguide and, in some embodiments, at least one of the cutting element and the light guiding element(s) may comprise an optical fibre. Providing a tapered portion in the optical waveguide or in the light guiding element may increase a numerical aperture of light propagating therethrough, which may increase the effectiveness of the light for cutting hair.

The at least one light source may, according to some embodiments, comprise a first light source for generating light having the first wavelength and a second light source for generating light having the second wavelength.

The at least one light source may be configured to generate light having the first wavelength at a relatively lower intensity, and the series of pulses of light having the second wavelength at a relatively higher intensity. The lower intensity light is less likely to cause a large temperature increase in the optical waveguide. Thus, using low intensity light is less likely to result in damage being caused to the optical waveguide. The higher intensity light may be more effective in initiating cutting of the hair. By providing the high intensity light only in short-duration pulses, large temperature increases in the optical fibre may be prevented.

In some embodiments, the first wavelength may relatively longer than the second wavelength. For example, the first wavelength may be above 700 nm and/or the second wavelength may be between around 400 nm and around 500 nm.

The hair cutting device may comprise a sensor for detecting the presence of an object in contact with the cutting element. The at least one light source may be configured to generate a pulse of the series of pulses of light having the second wavelength in response to the detection of an object in contact with the cutting element. In this way, the pulses of high intensity light are generated only when necessary, resulting in a reduction in energy expenditure. The sensor may be a grating, such as a fibre Bragg grating.

According to a second aspect, there is provided a method of operating a hair cutting device, the hair cutting device having at least one light source and a cutting element for receiving light from the at least one light source. The method comprises generating light having a first wavelength and delivering the laser light having a first wavelength to the cutting element; and generating a first pulse of light having a second wavelength, and delivering the first pulse of laser light to the cutting element.

According to some embodiments, the method may further comprise generating a second pulse of light having the second wavelength, and delivering the second pulse of laser light to the cutting element after a defined time has elapsed following the delivery of the first pulse.

In some embodiments, the method may comprise detecting, using a sensor, the presence of an object in contact with the cutting element. Upon detection of the presence of an object in contact with the cutting element, the method may comprise delivering the first pulse of light having a second wavelength to the cutting element.

During delivery of a pulse of light having the second wavelength, the at least one light source may be prohibited from generating light having the first wavelength. This may result in an energy saving as light having the two different wavelengths is not generated concurrently.

The first wavelength may be relatively longer than the second wavelength. The light having the first wavelength may have a relatively lower intensity and/or light having the second wavelength may have a relatively higher intensity.

Other advantageous features will become apparent from the following description.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a block diagram of a hair cutting device according to embodiments of the invention;

FIG. 2 is a pair of schematic drawings showing different views of an exemplary hair cutting device according to embodiments of the invention;

FIG. 3 is a graph illustrating the refractive index of hair;

FIG. 4 is a graph showing laser light generation durations according embodiments of the invention; and

FIG. 5 is a graph showing laser light generation durations according alternative embodiments of the invention.

### DETAILED DESCRIPTION OF THE EMBODIMENTS

As noted above, the present invention provides an improvement in the longevity of a laser light-based shaving device, for example as described in WO 2014/143670. In particular, it has been recognised that the likelihood of damaging a cutting element may be reduced by delivering a relatively low intensity of light to the cutting element. By reducing the chance of the cutting element overheating and, therefore, becoming damaged, the useful life of the cutting element and the hair cutting device to which the cutting element is fitted are likely to be lengthened. Consequently, the need for a user to replace the cutting element or the hair cutting device is reduced.

It will be appreciated that the invention is applicable to shaving devices (e.g. razors or electric shavers), and any other type of device that is used to cut hair (e.g. hair clippers), even if those devices do not necessary aim to provide a ‘clean shave’ (i.e. to remove hair at the level of the skin).

As discussed below, the cutting element may, in some examples, include an optical waveguide, such as an optical fibre. Light propagating through the optical fibre may couple out of the fibre into a surrounding medium, forming an evanescent field. When a hair is brought into contact with or placed in very close proximity to the fibre, it may interact with the evanescent field surrounding the fibre and thereby cause light being transmitted from the fibre to the hair. This interaction may initiate cutting or melting of the hair. In order to ensure effective cutting, the interaction of the hair with the light field around the fibre should be such that significant out-coupling is ensured. It has been discovered that providing a tapered portion (i.e. a portion having a reduced diameter) in the optical fibre may improve the cutting element efficiency. Using tapering, a numerical aperture (NA) of the guided beam may be increased and the tapering ratio can be chosen such that it is close or equal to the maximum NA supported by the fibre, effectively maximizing the extent of the evanescent field and therewith the interaction depth of the evanescent field within the hair.

The inventors realized that using a tapered portion to increase the NA of the beam in the fibre might provide penetration depths which otherwise could only have been achieved by using much more powerful laser sources. Allowing less powerful laser sources to be used in the hair cutting device not only improves the energy efficiency of the hair cutting device, but also contributes to the overall safety of the hair cutting device.

The inventors realized that a further effect of increasing the NA of the light beam through the fibre increases the intensity incident on an object touching the fibre, such as a hair that is placed in contact with the fibre, in itself. A light ray travelling with a higher NA will have more interactions with the edge of the fibre per unit of length compared to a light particle travelling with a lower NA through the same fibre. As an example, assuming that the hair is a relatively large object compared to the thickness of the light-guide, it can be noted that a single light-ray has a certain opportunity to interact with the hair whereby the likelihood that it does is larger for the high NA light because it will strike the fibre surface at a higher rate, effectively increasing the probability for interaction (in other words, the absorption cross-section of the hair is effectively increased).

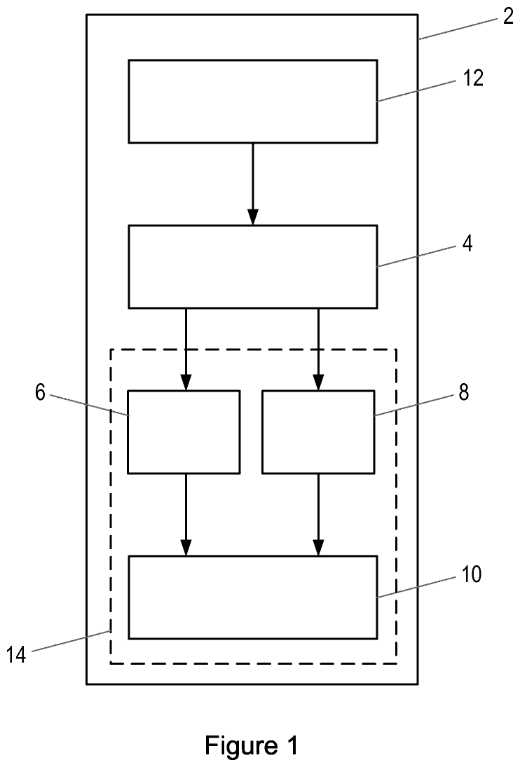

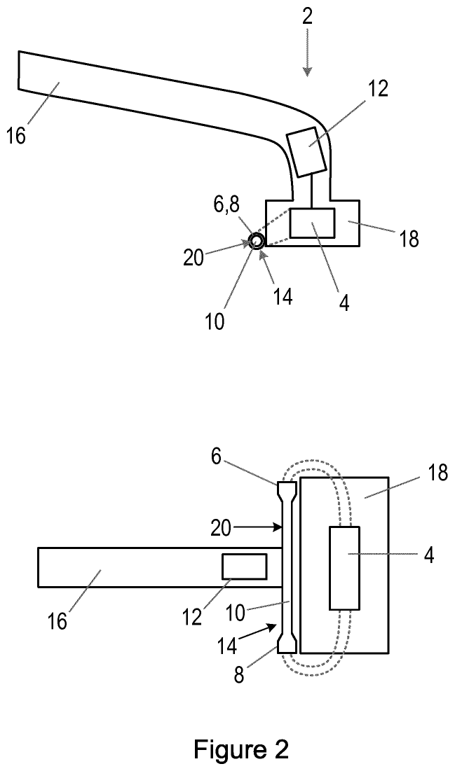

FIG. 1 is a block diagram of a hair cutting device 2 according to an embodiment of the invention. FIG. 2 shows a hair cutting device 2 in the form of a handheld razor according to an exemplary embodiment of the invention. The hair cutting device 2 is for cutting (e.g. shaving) hair on a body of a subject. The subject may be a person or an animal. The hair may be facial hair (i.e. hair on the subject's face), or hair on the subject's head or other part of their body (legs, chest, etc.).

Referring to FIG. 1, the hair cutting device 2 comprises at least one light source 4, such as a laser light source, for generating light at two or more specific wavelengths corresponding to wavelengths absorbed by one or more chromophores in hair. As discussed below, the at least one light source 4 may comprise a single light source or multiple light sources. The light emitted by the at least one light source 4 is, in this example, coupled into a first light guiding element 6 and a second light guiding element 8 which guide the light towards a cutting element 10. In some embodiments, the light emitted by the at least one light source 4 may be coupled into a single light guiding element configured to direct the light towards the cutting element 10. In other embodiments, the light emitted by the at least one light source 4 may be coupled directly into the cutting element 10 without passing through one or more light guiding elements. The cutting element 10 enables hair to be cut as the hair cutting device 2 is moved over the skin of a subject. The cutting element 10 is an optical waveguide 10 that is arranged on the hair cutting device 2 so that the optical axis of the optical waveguide 10 (i.e. the line along which light typically propagates through the optical waveguide 10) is generally perpendicular to the direction in which the hair cutting device 2 is moved so that hairs contact the sidewall of the optical waveguide 10 (the sidewall corresponding to the long edge of the optical waveguide 10) as the hair cutting device 2 is moved across the skin of the subject. In some embodiments, the optical waveguide 10 is an optical fibre, although those skilled in the art will be aware of other types of optical waveguide that can be used according to the invention, such as a slab waveguide, a strip waveguide or a photonic crystal waveguide. An optical fibre comprises a core, and in some embodiments also comprises a cladding, which may or may not fully encompass the core (e.g. part of the core may be exposed).

The light (which in some embodiments may be laser light) that is generated by the at least one light source 4 (which in some embodiments may be one or more laser light sources) is coupled into the optical waveguide 10. In some embodiments, the light is coupled into only one end of the optical waveguide 10, which in other embodiments, the light may be coupled into both ends of the optical waveguide 10 so that the laser light propagates through the optical waveguide 10 from both ends.

The at least one light source 4 is configured to generate light (which, in this example, is laser light) at two or more specific wavelengths that can be used to cut or burn through hair. In particular, each wavelength corresponds to the wavelength of light absorbed by a chromophore that is found in hair. As is known, a chromophore is the part of a molecule that provides the molecule with its colour. Thus, the laser light will be absorbed by the chromophore and converted into heat which will melt or burn the hair or otherwise destroy the bonds in the molecules of the hair, and it is this melting or burning that provides the cutting action of the hair cutting device 2.

Suitable chromophores that can be targeted by the laser light generated by the at least one light source 4 include, but are not limited to, melanin, keratin and water. Suitable wavelengths of laser light that can be used include, but are not limited to, wavelengths selected from ranges such as 380 nm (nanometres) to 1000 nm (corresponding, for example, to melanin absorption), above 1200 nm (corresponding, for example, to water absorption) and 2700 nm to 3500 nm. Those skilled in the art will be aware of the wavelengths of light that are absorbed by these chromophores, and thus also the specific wavelengths of light that the at least one light source 4 should generate for this purpose, and further details are not provided herein.

In some embodiments the at least one light source 4 can be configured to generate laser light at a plurality of wavelengths (either simultaneously or sequentially), with each wavelength being selected to target a different type of chromophore. This can improve the cutting action of the optical waveguide 4 since multiple types of molecules in the hair may be burnt using the laser light. In embodiments which include multiple light sources 4, each light source may generate laser light at a respective wavelength, and each light source can be coupled to a single optical waveguide 10 or to a respective optical waveguide to provide multiple cutting elements 10 in the device 2.

In some embodiments, the hair cutting device 2 also comprises a control unit 12 that controls the operation of the hair cutting device 2, and in particular is connected to the at least one light source 4 to control the activation and deactivation of the at least one light source 4 (and in some embodiments control the wavelength and/or intensity of the light generated by the at least one light source 4). The control unit 12 may activate and deactivate the at least one light source 4 in response to an input from a user of the hair cutting device 2. The control unit 12 can comprise one or more processors, processing units, multi-core processors or modules that are configured or programmed to control the hair cutting device 2.

The optical waveguide 10 (i.e. the cutting element) and the first and second light guiding elements 6, 8 (or single light guiding element) may be considered to form components of a cutting assembly 14 which, in some embodiments, may be detachable from the hair cutting device 2, and may be enclosed in a separate unit or housing.

As noted above, FIG. 2 shows a hair cutting device 2 that is in the form of a handheld wet razor. FIG. 2 shows a side view and a bottom view of the razor 2. The razor 2 comprises a handle 16 for the subject (or other user of the device 2) to hold, and a head portion 18 that includes the cutting element 10 (optical waveguide/fibre). As shown, the optical waveguide 10 is arranged along an edge of the head portion, and a part of the optical waveguide 10 forms (or corresponds to) a cutting face 20. The cutting face 20 is the part of the optical waveguide 10 that is intended to come into contact with hair as the hair cutting device 2 is moved across the skin of the subject. Light from the at least one light source 4 is directed or guided into the cutting element via the light guiding element(s). In the example shown in FIG. 2, the hair cutting device includes two light guiding elements. In this example, therefore, light from the at least one light source 4 is directed or guided into a first end of the optical waveguide 10 via the first light guiding element 6, and into a second end of the optical waveguide 10 via the second light guiding element 8. In other embodiments, a single light guiding element may be provided at one end of the cutting element 10, and may be configured to direct light from the at least one light source 4 into one end of the optical waveguide.

The at least one light source 4 and the control unit 12 are shown as being incorporated into the head portion 18 and handle 16 respectively, but it will be appreciated that the positions of these components in the hair cutting device 2 as shown in FIG. 2 is not limiting. In embodiments in which the at least one light source comprises two or more light sources, the two or more light sources may both be incorporated into the head portion 18 or the handle 16, or located at various positions on or in the hair cutting device 2. Likewise it will be appreciated that the embodiment shown in FIG. 2 is merely an example, and the invention can be incorporated or used in any type of hair cutting device 2 that comprises an optical waveguide cutting element 10 as described herein. As noted above, the optical waveguide 10 and the light guiding element(s) (the first light guiding element 6 and the second light guiding element 8 in this example) may form part of a cutting assembly 14 which may itself form a part of, or be detachably connected to, the head portion 18.

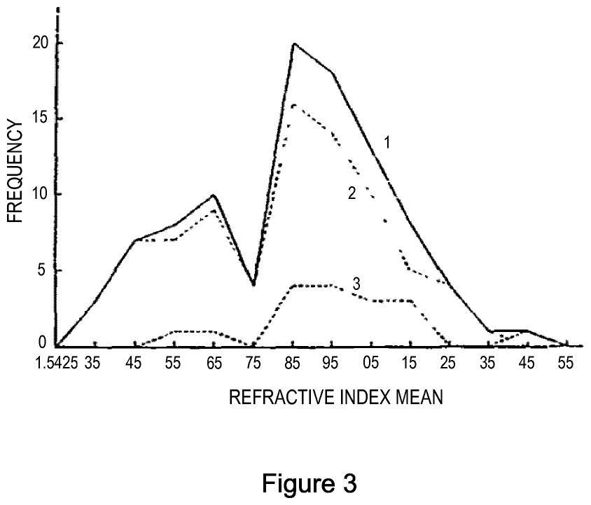

The graph in FIG. 3 illustrates the refractive index of hair, which can be found in a paper by M. D. Greenwell, A. Willner, Paul L. Kirk: Human Hair Studies: III. Refractive Index of Crown Hair, 31 Am. Inst. Crim. L. & Criminology 746 (1940-1941). Curve 1 is a composite line, curve 2 is a line representing the refractive index for Caucasian people, and curve 3 is a line representing the refractive index for non-Caucasian people. Thus, it can be seen that the refractive index of hair is between (approximately) 1.545 and 1.555, although there will be variation between individuals. For example the above paper also recognises that the refractive index of hair can depend on the sex of the subject, e.g. the refractive index of hair on a female is generally higher than the refractive index of hair on a male.

As is known, the light guiding element(s) and the cutting element 10 together act as a waveguide for the light coupled from the one or more light sources 4 through the occurrence of total internal reflection, since the refractive index of air is lower than that of the optical waveguide. However, if an object that has a refractive index higher than the optical waveguide is put into contact with the cutting element 10, then the total internal reflection is ‘frustrated’ and light can couple from the optical waveguide into that object. Thus, in order for light to be coupled into a hair from the cutting element 10 part of the optical waveguide (to provide the cutting action according to the invention), the optical waveguide must have the same or a lower refractive index than hair at the point at which the hair contacts the cutting element 10. Thus, the optical waveguide must have the same or a lower refractive index than hair at least at the cutting face 20 portion of the cutting element. Preferably, the refractive index of the optical waveguide at the cutting face 20 is the same as that of hair since that provides the best coupling of light from the optical waveguide to the hair.

Thus, in some embodiments, the refractive index of the optical waveguide 10 at least at the cutting face 20 is equal to or lower than 1.56. More preferably the refractive index of the optical waveguide 10 at least at the cutting face 20 is equal to or lower than 1.55. Even more preferably, the refractive index of the optical waveguide 10 at least at the cutting face 20 is equal to or lower than 1.54, since this refractive index is below the refractive indices identified in FIG. 3.

In some embodiments, a lower bound for the refractive index of the optical waveguide 10 at the cutting face 20 can be 1.48, 1.51, 1.53 or 1.54.

A range of values from which the refractive index of the optical waveguide 10 is selected can be formed from any combination of the upper and lower refractive index bounds set out in the preceding paragraphs.

The optical waveguide/fibre 10 can be made from any suitable material or combination of materials. For example optical waveguides/fibres can be composed of or comprise silica, fluoride glass, phosphate glass, chalcogenide glass, crown glass (such as BK7) and/or crystals (such as sapphire or yttrium aluminium garnet (YAG)).

As noted above, light generated by the at least one light source 4 propagates through the optical waveguide 10 and, may couple out into a hair brought close to, or into contact with, the optical waveguide. For the light to initiate cutting or melting of the hair, the light should be of sufficient intensity/power. It has been recognised, however, that as light couples out of the optical waveguide 10 and into a hair, heat from the hair may rapidly transfer to the optical waveguide resulting in rapid increase in the temperature of the optical waveguide in localised regions or along its entire length. If the temperature of the optical waveguide increases too much, or to a critical temperature, the optical waveguide may be damaged. In some examples, the temperature of the optical waveguide may be caused to increase to near a glass transition temperature, T_{g}, of the optical waveguide. The glass transition temperature of an object is the temperature at which, or the range of temperatures over which, an amorphous material, such as glass, transitions from a hard, brittle state into a molten, rubber-like state. The glass transition temperature for an object depends on the material from which the object is made. In other words, different materials have different glass transition temperatures. If the temperature of the optical waveguide 10 is increased repeatedly to its glass transition temperature, then then waveguide may become brittle, and may break. It has been recognised that one way to reduce the chance of the optical waveguide becoming brittle, and breaking, is to prevent the waveguide from reaching its glass transition temperature.

According to some embodiments, the invention may be realised by limiting the time that high power or high intensity light propagates through the optical waveguide 10. The at least one light source 4 may generate light having two or more specific wavelengths corresponding to wavelengths absorbed by one or more chromophores in hair. The at least one light source 4 is configured to generate: (i) light having a first wavelength, and (ii) a series of pulses of light having a second wavelength. In some embodiments, the light having the first wavelength and/or the light having the second wavelength may be delivered to the cutting element via one or more light guiding elements, such as light guiding elements 6, 8, which may include one or more taper transition sections in which a diameter of the light guiding element (or of the cutting element) reduces from a first diameter to a second diameter. The light having the first wavelength may, in some embodiments, be continuous wave (CW) light, emitted constantly, for example without interruption. The light having the second wavelength may be generated in pulses. In some embodiments, light having the first wavelength and light having the second wavelength may be generated by a single light source 4, while, in other embodiments, the at least one light source 4 may comprise a first light source for generating light having the first wavelength and a second light source for generating light having the second wavelength. By generating light having multiple wavelengths from a single light source, component costs and manufacturing costs may be kept relatively low as only one light source may be incorporated into the hair cutting device 2. However, by using two separate light sources to generate light having the first and second wavelengths, a broader range of wavelengths may be achievable, and the light sources may be controlled to generate light having the two different wavelengths simultaneously.

As noted above, the light having the first and second wavelengths may be delivered to the cutting element 10 according to different timing schedules. The first wavelength and the second wavelength may be selected based on the type and/or colour of hair to be cut; it is known that light having particular wavelengths are absorbed by particular chromophores in the hair and, since chromophores may be present at particular locations within a strand of hair, light having particular wavelengths of light may penetrate more deeply into a strand of hair than light of other wavelengths. For example, light having a relatively long wavelength (e.g. greater than around 700 nm) may penetrate more deeply into a hair (i.e. the absorption coefficient at relatively longer wavelengths is lower, resulting in an increased penetration depth of the light) than light having a relatively shorter wavelength. Similarly, light having a relatively shorter wavelength (e.g. less than around 600 nm or, preferably, between around 400 nm and 500 nm) may be absorbed more readily by chromophores located near to the surface of a strand of hair than light having a relatively longer wavelength.

According to some embodiments of the invention, light having the first wavelength (i.e. a relatively longer wavelength) may be generated at a relatively low power or low intensity as continuous wave. The low intensity light may, in some embodiments, be of sufficient intensity to initiate cutting of some strands of hair, but may be low enough that the temperature of the optical waveguide 10 is not caused to increase to near its glass transition temperature. In other embodiments, the intensity of the light having the first wavelength may be lower than the intensity required to initiate cutting of a hair, but sufficient to continue the cutting process once initiated (for example by some other means, such as light having a different wavelength).

The light having the second wavelength (i.e. the relatively shorter wavelength) may be generated at a relatively high power or high intensity as a series of pulses. The high intensity light may, in some embodiments, be sufficient to initiate cutting of hair. By delivering the high intensity light as a pulse (i.e. over a very short duration), the light is able to couple out of the optical waveguide 10 into a hair, but the temperature of the hair is not caused to increase for a long duration. Thus, the temperature of the optical waveguide is not caused to increase to near its glass transition temperature as a result of the pulse of high intensity radiation. The pulse duration of each pulse (i.e. the duration that the at least one light source is active to generate a single pulse) of high intensity light may be between around 0.001 millisecond (ms) and 1 ms and, more preferably, between around 0.01 ms and 0.1 ms. The pulse repetition rate (i.e. the number of pulses emitted per second) may be between around 500 Hz and 50 kHz. In some embodiments, the pulse repetition rate may be based on a thermal relaxation time of the optical waveguide 10. In some examples, the thermal relaxation time of an object may be considered to be the time taken for the temperature of the object being heated to reduce by around 63% or 50% after the heating of the object (e.g. by the pulse of light) has ended. By basing the pulse repetition rate on the thermal relaxation time of the optical waveguide 10, which depends on the material from which the optical waveguide is constructed, an optical waveguide heated by a first light pulse is allowed to cool before a subsequent light pulse is generated. The temperature of the optical waveguide 10 is, consequently, kept below its glass transition temperature, thereby reducing the risk of damage. Thus, in general, following the delivery of a first pulse of light, a second pulse of light having the second wavelength may be generated and delivered to the cutting element after a defined time has elapsed following the delivery of the first pulse.

The combination of the application of high intensity pulses of light and a low intensity stream of light provides effective cutting and reduces the risk of damage occurring to the cutting element 10. The high intensity, short duration pulse of light having a relatively low wavelength is effective for initiating the cutting or melting of a hair, as the light is absorbed well near to the surface of the strand of hair. The low intensity stream of light having a relatively higher wavelength is effective for continuing and maintaining the cutting of the strand of hair, as the light is absorbed well deeper within the strand of hair.

In some embodiments, the light having the first wavelength may correspond to a relatively lower absorption coefficient of hair (and, therefore, may have a greater optical penetration depth), and the light having the second wavelength may correspond to a relatively higher absorption coefficient of hair (and, therefore, may have a shorter optical penetration depth). Thus, in some examples, light having a shorter wavelength may penetrate deeper into a strand of hair than light having a longer wavelength.

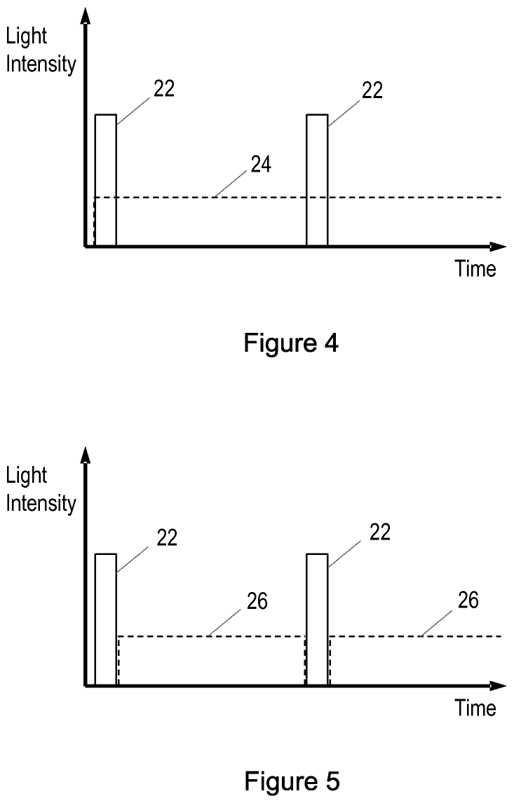

FIG. 4 is a graph showing a temporal profile of light generated by the at least one light source 4 according to some embodiments. The graph of FIG. 4 shows the relative light intensities of the light having the first wavelength and the light having the second wavelength over time. Pulses 22 of high intensity light having the second wavelength are shown spaced apart by a defined time which, as noted above, may be based on the thermal relaxation time of the optical waveguide 10. A continuous wave stream 24 of low intensity light having the first wavelength is also shown. In this example, the low intensity light having the first wavelength is generated and delivered to the optical waveguide 10 constantly, even during the generation of the high intensity pulses 22.

As noted above, the low intensity light having the first wavelength may, in some embodiments, be generated as a continuous wave, and delivered continuously into the optical waveguide 10. In other embodiments, however, the low intensity light having the first wavelength may be generated only when pulses of light having the second wavelength are not being generated. In other words, the at least one light source may be configured such that light having different wavelengths are not generated concurrently. Preventing the concurrent generation of light having multiple wavelengths may have power-saving benefits.

FIG. 5 is a graph showing a temporal profile of light generated by the at least one light source 4 according to alternative embodiments. In the graph of FIG. 5, the pulses 22 indicate that high intensity light having the second wavelength is generated at regular intervals. However, in this example, low intensity light having the first wavelength is not generated while the high intensity pulses are generated and, therefore, the low intensity light having the second wavelength is generated as a series of longer duration pulses 26 interleaved between adjacent high intensity pulses 22. Thus, during delivery of a pulse of light having the second wavelength, the at least one light source may be prohibited from generating light having the first wavelength. The control unit 12 may control the at least one light source to generate light or prevent the generation of light.

In some embodiments, the generation of pulses 22 may be performed in response to a trigger signal, rather than at regular time intervals. For example, the hair cutting device 2 may further include a sensor for detecting the presence of an object in contact with the cutting element 10. The sensor may detect when the optical waveguide 10 comes into contact with a hair, for example and, in response to such a detection, the at least one light source may generate a pulse of light having the second wavelength. In this way, pulses of high intensity light may be generated only when needed to initiate cutting of a strand of hair, thereby reducing energy expenditure.

The sensor used to detect the presence of an object in contact with the cutting element 10 may be a sensor associated with the control unit 12 of the device 2, and may be located in or on the device, or remote from the device. In some embodiments, the sensor may comprise a sensor located within the optical waveguide 10 or within one or more of the light guiding elements 6, 8. The sensor may, in some embodiments, comprise a grating, such as a fibre Bragg grating, which may be configured to deform upon contact with an object, and generate a signal based on the nature of the object coming into contact with the optical waveguide 10.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage.

Any reference signs in the claims should not be construed as limiting the scope.

1. A hair cutting device for cutting hair on a body of a subject, the hair cutting device comprising:
at least one light source for generating light at two or more specific wavelengths corresponding to wavelengths absorbed by one or more chromophores in hair; and
a cutting element comprising an optical waveguide for receiving light from the at least one light source;
wherein the optical waveguide comprises a cutting face, the cutting face being arranged to contact hair as the hair cutting device is moved across the skin of the body of a subject,
wherein the cutting face is arranged essentially parallel to the long axis of the optical waveguide,
wherein the optical waveguide is arranged to allow the light generated by the at least one light source to couple into hair when hair is close to or in contact with the optical waveguide; and
wherein the at least one light source is configured to generate light having a first wavelength and a series of pulses of light having a second wavelength.

2. A hair cutting device according to claim 1, further comprising:
a first light guiding element configured to guide light from the at least one light source into a first end of the cutting element; and
wherein the first light guiding element comprises a taper transition section in which a diameter of the light guiding element reduces from a first diameter to a second diameter.

3. A hair cutting device according to claim 2, further comprising:
a second light guiding element configured to guide light from the at least one light source into a second end of the cutting element;
wherein the second light guiding element comprises a taper transition section in which a diameter of the light guiding element reduces from a third diameter to a fourth diameter.

4. A hair cutting device according to claim 1, wherein the at least one light source comprises:
a first light source for generating light having the first wavelength; and
a second light source for generating light having the second wavelength.

5. A hair cutting device according to claim 1, wherein the at least one light source is configured to generate light having the first wavelength at a relatively lower intensity, and the series of pulses of light having the second wavelength at a relatively higher intensity.

6. A hair cutting device according to claim 1, wherein the first wavelength is relatively longer than the second wavelength.

7. A hair cutting device according to claim 1, wherein the first wavelength is above 700 nm.

8. A hair cutting device according to claim 1, wherein the second wavelength is between around 400 nm and around 500 nm.

9. A hair cutting device according to claim 1, further comprising:
a sensor for detecting the presence of an object in contact with the cutting element;
wherein the at least one light source is configured to generate a pulse of the series of pulses of light having the second wavelength in response to the detection of an object in contact with the cutting element.

10. A method of operating a hair cutting device, the hair cutting device having at least one light source and a cutting element for receiving light from the at least one light source, the method comprising:
generating light having a first wavelength and delivering the laser light having a first wavelength to the cutting element; and
generating a first pulse of light having a second wavelength, and delivering the first pulse of laser light to the cutting element.

11. A method according to claim 10, further comprising:
generating a second pulse of light having the second wavelength, and delivering the second pulse of laser light to the cutting element after a defined time has elapsed following the delivery of the first pulse.

12. A method according to claim 10, further comprising:
detecting, using a sensor, the presence of an object in contact with the cutting element;
upon detection of the presence of an object in contact with the cutting element, delivering the first pulse of light having a second wavelength to the cutting element.

13. A method according to claim 10, wherein, during delivery of a pulse of light having the second wavelength, the at least one light source is prohibited from generating light having the first wavelength.

14. A method according to claim 10, wherein the first wavelength is relatively longer than the second wavelength.

15. A method according to claim 10, wherein the light having the first wavelength has a relatively lower intensity, and light having the second wavelength has a relatively higher intensity.
